(19) United States
(12) Plant Patent Application Publication
(10) Pub. No.: US 2023/0007822 P1
(43) Pub. Date: Jan. 5, 2023

(54) Thuja PLANT NAMED 'RutThu4'

(50) ;

(71) Applicant: University of Georgia Research Foundation, Inc., Athens, GA (US)

(72) Inventor: John M. Ruter, Watkinsville, GA (US)

(21) Appl. No.: 17/854,214

(22) Filed: Jun. 30, 2022

Related U.S. Application Data
(60) Provisional application No. 63/217,004, filed on Jun. 30, 2021.

(51) Int. Cl. A01H 7/00

(52) U.S. Cl. PLT/213

(57) ABSTRACT

A new and distinct cultivar of Thuja plant named ‘RutThu4’, characterized by its compact, globose growth, similar in shape but larger in size and with a faster growth rate than Thuja occidentalis ‘Little Giant’. ‘RutThu4’ also has disease resistance when grown in USDA Zone 8a, and has demonstrated both heat tolerance as well as cold hardiness to about −34 ° F. in trials conducted in Minnesota.

### CROSS-REFERENCE TO RELATED APPLICATIONS

This application claims priority to U.S. provisional application titled “Thuja PLANTS NAMED ‘RutThu4’, ‘RutThu3’, ‘RutThu4’, and ‘RutThu5’” having serial number 63/217,004, filed on Jun. 30, 2021, which is entirely incorporated herein by reference.

Botanical designation: Thuja species.

Cultivar denomination ‘RutThu4’.

### BACKGROUND OF THE INVENTION

The present invention relates to a new and distinct cultivar of Thuja, botanically know as Thuja occidentalis, and hereinafter referred to by the cultivar name ‘RutThu4’.

The new Thuja cultivar ‘RutThu4’ is a product of open pollination of Thuja occidentalis ‘Filiformis’ (unpatented). The open-pollinated seeds were obtained in 2010 from American Conifer Society. Seedlings were planted in Watkinsville in 2014 The new cultivar ‘RutThu4’ was selected for its compact, globose growth form, faster growth rate than Thuja occidentalis ‘Little Giant’, disease resistance in USDA Zone 8a, heat tolerance, and cold hardiness to about −34° F. in trials conducted in Minnesota.

Asexual reproduction of the new Thuja ‘RutThu4’ using hardwood cuttings has been continued in Watkinsville, Ga. since 2015. Observations of the resulting progeny have shown that the unique features of this new Thuja ‘RutThu4’ are stable and reproduced true to type in successive generations. The new ‘RutThu4’ has also been grown in MN.

### SUMMARY OF THE INVENTION

The new Thuja cultivar ‘RutThu4’ has not been observed under all possible environmental conditions. The phenotypes may vary somewhat with variations in environment and cultural practices such as temperature, water and fertility levels, soil types, and light intensity without, however, any variance in genotype.

The following traits have been repeatedly observed and are determined to be the unique and distinguishing characteristics of the new Thuja cultivar named ‘RutThu4’. In combination, these traits set ‘RutThu4’ apart from all other existing varieties of Thuja known to the inventors.
1. compact, globose growth form;
2. faster growth rate than Thuja occidentalis ‘Little Giant’;
3. resistance to diseases common to other Thuja cultivars in USDA zone 8a;
4. heat tolerance
5. cold hardiness to about −34° F. in trials conducted in Minnesota.

Plants of the ‘RutThu4’ can be compared to similar cultivar Thuja occidentalis ‘Little Giant’ (unpatetend) differs from ‘Little Giant’ in that ‘RutThu4’ has a similar rounded shape as ‘Little Giant’ while being larger (both talker and wider) than ‘Little Giant’ (which is about 36″×48″, as compared to about 58″×80″ for ‘RutThu4’). Male parent plant is not conclusively known, so comparison data is not available. The new Thuja ‘RutThu4’ can also be compared to Thuja occidentalis cultivars ‘Filiformis’ (female parent) in that ‘RutThu4’ has foliage typical of the species, unlike ‘Filiformis’.

### BRIEF DESCRIPTION OF THE PHOTOGRAPHS

The accompanying, colored photographic illustrations show the overall appearance and distinct characteristics of the new cultivar Thuja ‘RutThu4’ showing the colors as true as possible. Colors in the photographs may differ slightly from the color values cited in the detailed botanical description. The photographs were taken around June, 2021 of plants grown outdoors in Watkinsville, Ga.

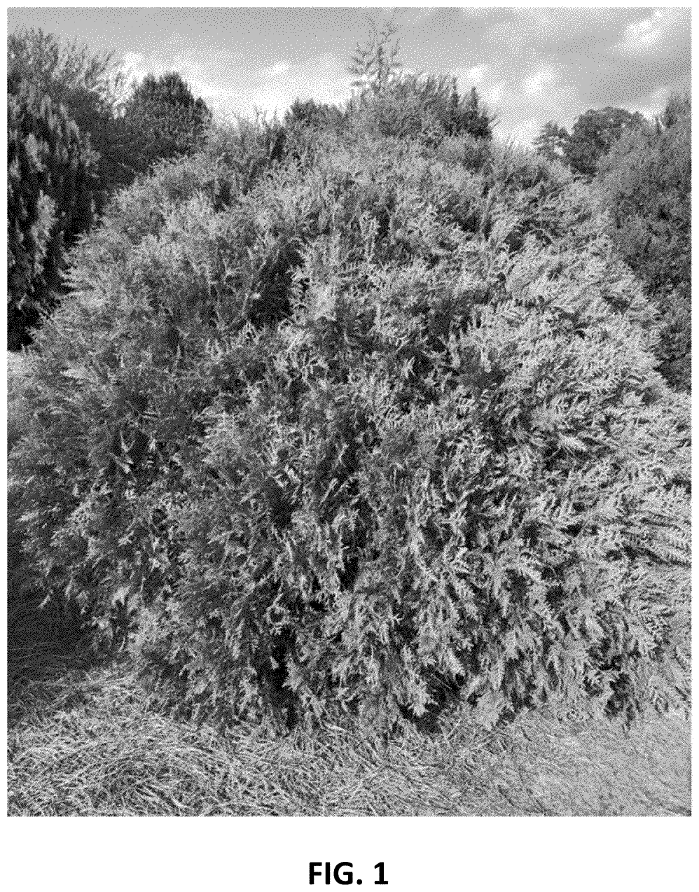

The photograph labeled FIG. 1 depicts a side view of a typical 7-year-old ‘RutThu4’ plant showing the rounded, compact globose form and features.

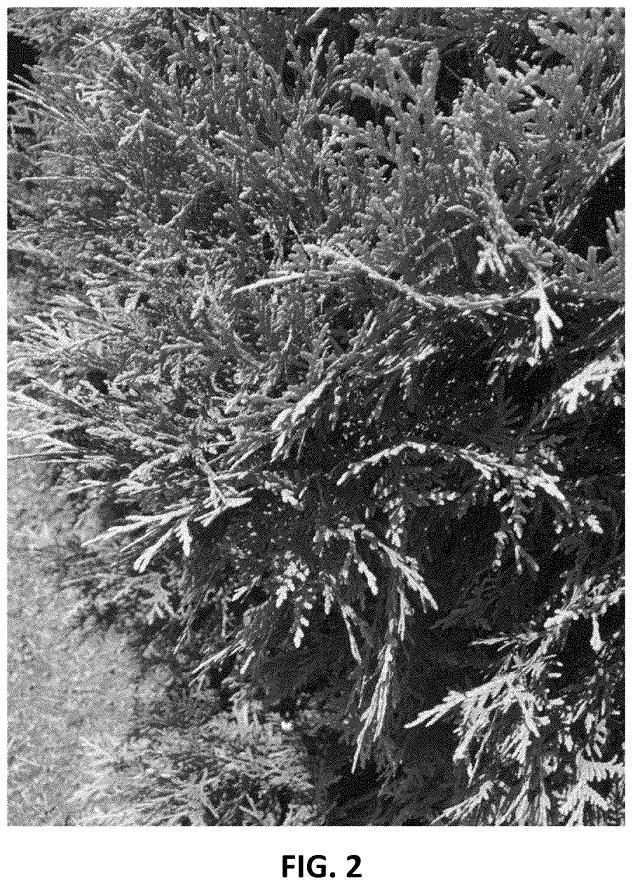

The photograph labeled FIG. 2 depicts a close-up view of the branches and needles of a typical 7-year-old ‘RutThu4’ plant.

### DETAILED BOTANICAL DESCRIPTION

The following traits have been consistently observed in the original plants of the new varieties and in asexually propagated progeny grown in Watkinsville, Ga., (and some in MN) and, to the best knowledge of the inventors, their combination forms the unique characteristics of the new variety ‘RutThu4’.

Throughout this specification, color names beginning with a small letter signify that the name of that color, as used in common speech, is aptly descriptive. Color names beginning with a capital letter designate values based upon The R.H.S. Colour Chart, 5th edition published by The Royal Horticultural Society, London, England in 2007, except where general terms of ordinary dictionary significance are used

The aforementioned photographs and following observations, measurements, and values describe plants of the Thuja cultivar named ‘RutThu4’. Data were collected from a horticulture farm in Watkinsville, Ga. from 6-year-old plants. The average low temperatures ranged from about 20° F. to 30° F. in the winter and about 90° F. to 100° F. in summer.
Botanical classification: Thuja ‘RutThu4’.
Commercial classification: Ornamental conifer.
Parentage: Open pollination of Thuja occidentalis ‘Filiformis’ by unknown pollen parents.
Growth and propagation:
   Propagation type.—Terminal or sub-terminal hardwood cuttings.
   Growth rate.—Slow to moderate.
   Root description.—Typically about 2-5 roots arising from the base of the cuttings.
   Rooting habit.—Good.
Plant description:
   Habit.—Of 6-year-old plant in field — compact round/globose, about symmetrical
   Usage.—Various uses, including landscaping, ornamental, container, border, hedge, and mass planting.
   Vigor.—Moderate.
   Size of 6-year-old plant.—A. Height: from soil level to top of plant, about 58 inches. B. Width: about 80 inches. C. Ratio: about 0.73.
   Branch/stem (terminal tuft).—A. Quantity: numerous. B. Length: about 13-14.5 cm. C. Base Diameter: about 2-3 mm at about 15 cm from tip. D. Branch angle: about 65° from center. E: Odor (of bruised stem): moderate evergreen scent. F. Internode length: about 1-1.5 mm (about 5 internodes per sample). G: Color (RHS): Greyed-orange 166A. H: Strength of lateral branches: moderate. I: Pubescence: has scale that peel off but do not shed. J: Shape: round.
   Leaf.—A. Leaf type: needles. B. Mature size (L×W): about 5-9 cm×about 2-3 cm. C. Arrangement on stem: alternate. D. Aspect: upright. E. Color (RHS): summer — upper and lower, Green 137B, tips have tinge of Yellow-Green 144C; winter — upper is Greyed-orange 165A; lower is Greyed-Orange 166D. F. Leaf Number: average of about 8 per cm/stem. G. Apex: acute. H. Base: cuneate. I. Margin: entire. J. Shape: linear with scale-like structure, no lobes present. K. Pubescence: none. L. Venation: none. M. Texture: smooth, glossy. 0. Odor when crushed: light evergreen scent.
   Coning production.—None.
   Seed.—None.
Weather/temperature tolerance: Plants of the new Thuja have been observed to grow in USDA Zone 8a, with heat tolerance, and have a USDA Zone range 3-8. ‘RutThu4’ has also been cold hardy to about −34° F. in trials conducted in Minnesota.
Disease/pest resistance: To date, plants of the new Thuja have been observed to have disease resistance in USDA Zone 8a when grown under standard conditions.

It is claimed:

1. A new and distinct cultivar of the Thuja plant named ‘RutThu4’ as illustrated and described herein.
